(19) United States
(12) Patent Application Publication
(10) Pub. No.: US 2007/0123781 A1
(43) Pub. Date: May 31, 2007

(54) Surgical anastomosis leak detection system

(75) Inventors: Mark J. Callahan, Westport, MA (US); Mark S. Guitarini, Amherst, MA (US)

Correspondence Address: CARTER, DELUCA, FARRELL & SCHMIDT, LLP, 445 BROAD HOLLOW ROAD, SUITE 225, MELVILLE, NY, 11704, US

(73) Assignee: Tyco Healthcare Group LP

(21) Appl. No.: 11/287,652

(22) Filed: Nov. 28, 2005

(51) Int. Cl. A61B 5/02

(52) U.S. Cl. 600/483

(57) ABSTRACT

A system for evaluating the integrity of an anastomosis includes a pressure source having a fluid conduit for insertion in the body and a sensor for measuring the rate of pressure decay of a predetermined amount of pressure supplied adjacent the anastomosis site.

### BACKGROUND

1. Technical Field

The present disclosure relates to methods and apparatii for verifying the integrity of an anastomosis resulting from various surgical procedures. More particularly, the present disclosure relates to a system and method for pressurizing an area inside tissue at an anastomosis site and measuring the rate of pressure change adjacent the anastomosis site.

2. Background Of Related Art

During various surgical procedures, it may become necessary to connect one tubular tissue section to another tubular tissue section. This type of surgical procedure is termed a surgical “anastomosis procedure”. An anastomosis procedure can be performed in one of two ways. The first is termed an “end to end” anastomosis procedure while the second is termed an “end to side” anastomosis procedure. An end to end anastomosis is typically performed during such surgeries as colorectal surgery and gastric bypass surgery. For example, during a colorectal surgery, it is necessary to remove a diseased section of the colon and reconnect the two healthy end sections of the colon. In gastric bypass surgery, a section of the stomach is bypassed to minimize the volume of the stomach and the intestine is shortened. The ends of the tissue sections are then reconnected.

An end to side anastomosis may be performed in conjunction with a heart bypass surgery. In this instance, it is necessary to connect one open end of a tubular tissue section to the side of another tubular tissue section.

Various methods and devices are provided for connecting sides or ends of tubular tissue sections. For example, one prior art method employs applying suture or staple lines to connect various tubular tissue sections. More complex devices include the use of single or multipart fasteners to secure the tubular tissue sections.

In most anastomosis procedures, it is often necessary to check for leaks, i.e., to verify the integrity of the anastomosis site, in order to ensure proper sealing of the tissue sections. Various methods are known to check the integrity of an anastomosis site and typically include the step of directly visualizing the anastomosis site. For example, in one method, a methylene blue dye is injected near the site and the anastomosis joint is visualized to monitor the escape of the dye, which would indicate the presence of leaks. However, in addition to the difficulty and inaccuracies resulting from attempting to verify the integrity by direct visualization, certain patients may be allergic to the dye. Further, direct visualization methods either with the naked eye or through the use of a laparoscope or an endoscope adds substantial time to the surgical procedure.

The presence of minor leaks at an anastomosis site is generally not considered serious as they will close during the normal healing process. However, more significant leaks may require additional surgical intervention involving extra time, effort and inconvenience to the patient. Therefore, it would be desirable to have a more efficient and accurate method of determining the integrity of an anastomosis site.

### SUMMARY

Accordingly, the present disclosure is directed to a system, apparatus and method for evaluating the integrity of an anastomosis site. In one preferred embodiment, a system for monitoring the integrity of an anastomosis of first and second tubular organ sections is disclosed. The system includes a pressure source for supplying fluids under pressure, first and second occluding members dimensioned for sealing a tubular organ section at first and second tissue sites adjacent respective opposed sides of an anastomosis site, a fluid conduit connectable to the pressure source and being adapted to deliver the fluids to the tubular organ sections between the first and second tissue sites, and a pressure sensor adapted to detect fluid pressure change adjacent the anastomosis site. At least one of the first and second occluding members includes a clamp which is positionable about the tubular organ section. Preferably, each of the first and second occluding members includes a clamp. The clamps are adapted for positioning about the tubular organ section adjacent the first and second tissue sites.

In one embodiment, the fluid conduit is adapted for passage through a lumen of the tubular organ section. The fluid conduit may include an insertion end portion adapted to form a substantial seal within the lumen of the tubular organ section adjacent one of the first and second tissue sites. Preferably, an expandable member, e.g., a balloon member, is mounted adjacent the insertion end portion thereof. The expandable member is expandable to form a substantial seal within the lumen of the tubular organ section. First and second expandable members may be provided to substantially seal the lumen adjacent the first and second tissue sites.

The fluid conduit may include an insertion needle associated therewith adapted to penetrate the tubular organ section to access the internal lumen thereof. The pressure sensor may be mounted to the fluid conduit. A controller may be associated with the pressure sensor. The controller includes logic to measure a rate of pressure loss adjacent the anastomosis site. Means for adjusting the sensitivity of the pressure sensor is also contemplated.

In another aspect of the disclosure, a method for monitoring the integrity of an anastomosis procedure is disclosed. The method includes the steps of sealing a tubular organ section at first and second tissue sites adjacent respective opposed sides of an anastomosis site, introducing fluids adjacent the anastomosis site between the first and second tissue sites and monitoring pressure change adjacent the anastomosis site with a pressure sensor. The step of monitoring may include introducing the pressure sensor within the tubular organ section between the first and second tissue sites. The step of monitoring may include monitoring pressure loss or a rate of pressure loss.

In one embodiment, the step of sealing includes applying a clamp adjacent at least one of the first and second tissue sites, preferably, adjacent each of the first and second tissue sites.

The step of introducing fluids includes positioning a fluid conduit into a lumen at the tubular organ section between the first and second tissue sites. The fluid conduit is in fluid communication with a pressure source and is adapted to deliver fluids under pressure to the tubular organ section.

The step of sealing may include positioning an end portion of the fluid conduit within the lumen of the tubular organ section and forming a substantial seal within the lumen of the tubular organ section with the end portion. The end portion of the fluid conduit may have an expandable member coaxially mounted thereabout. The expandable member is expanded to form a substantial seal within the lumen of the tubular organ section. The expandable member may include a balloon member. The fluid conduit may include a pressure sensor which is positioned within the tubular organ section between the first and second tissue sites.

### BRIEF DESCRIPTION OF THE DRAWINGS

Various embodiments of the presently disclosed system, apparatus and method for monitoring the integrity of an anastomosis site are disclosed herein with reference to the drawings, wherein:

FIG. 1 is a perspective view of a prior art apparatus and method for monitoring the integrity of an anastomosis site;

FIG. 2 is a perspective view, partially shown in section, of the system and method for monitoring the integrity of an end to end anastomosis site in accordance with the principles of the present disclosure;

FIG. 2A is a side view, shown in section, of the distal end of the fluid conduit of the system of FIG. 2;

FIG. 3 is perspective view of an alternate embodiment of the system and method of the present disclosure;

FIG. 4 is a perspective view illustrating use of the system of FIG. 3 in monitoring the integrity of an end to side anastomosis site; and

FIG. 5 is perspective view of another alternate embodiment of the system and method of the present disclosure.

### DETAILED DESCRIPTION OF THE PREFERRED EMBODIMENT(S)

Embodiments of the presently disclosed system and method for evaluating the integrity of an anastomosis site will now be described in detail with reference to the drawings wherein like numerals designate identical or corresponding elements in each of the several views. As is common in the art, the term proximal refers to that part or component closer to the user or operator, i.e. surgeon or physician, while the term distal refers to that part or component more remote from the user.

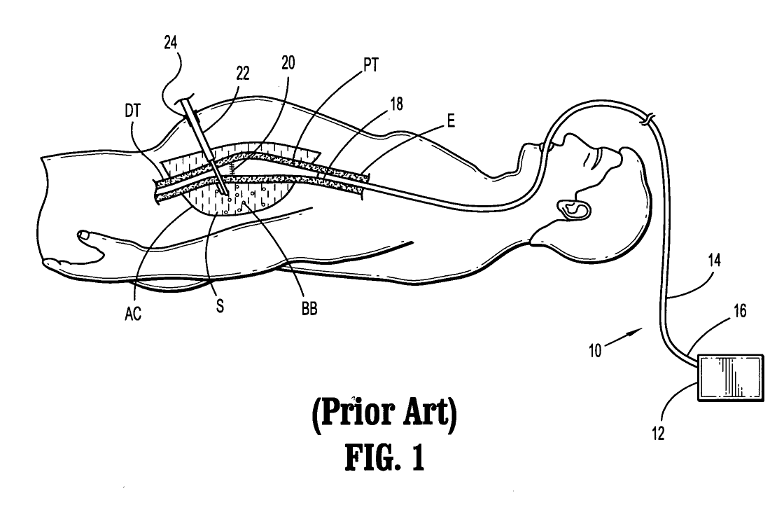

FIG. 1 illustrates a prior art method and apparatus for monitoring the integrity of an anastomosis site. Prior art device 10 includes pressure source 12 and pressure line 14. Pressure line 14 includes proximal end 16 which is connected to pressure source 12. In using this prior art device, distal end 18 of pressure line 14 is inserted intraorally and advanced within the esophageal tract to a location adjacent anastomosis site 20. For illustrative purposes, anastomosis site 20 is an end to end anastomosis performed during a gastric bypass procedure as discussed hereinabove. Clamp 22 is inserted through port 24 to clamp a distal tissue section DT distal of anastomosis site 20. Distal end 18 of pressure line 14 disposed within esophagus E forms an internal airtight seal with a proximal tissue section PT located proximal to anastomosis site 20.

The method further includes filling the abdominal cavity AC with saline S and activating the pressure source 12 such that the area of anastomosis site 20 between clamp 22 in distal tubular tissue DT and distal end 18 of pressure line 14 located within proximal tissue PT is pressurized. Once anastomosis site 20 has been pressurized, a laparoscope may be inserted through a second port (not shown) and the air bubbles BB escaping through the anastomosis site 20 and into saline S are visualized through the laparoscope. This known method is fairly invasive due to the insertion of the endoscope, and cumbersome due to the time required to evaluate the degree of bubbles BB escaping from anastomosis site 20. Additionally, due to the inconsistencies in the quantity of bubbles escaping through anastomosis site 20, it is difficult to obtain a precise determination of the degree of actual leakage through anastomosis site 20. As noted above, the presence of minor leaks would not be problematic, while the presence of a larger degree of leaks would require further surgical intervention. Thus, the prior art methods, which rely on visualization of air bubbles to evaluate the integrity of anastomosis site 20, are imprecise at best.

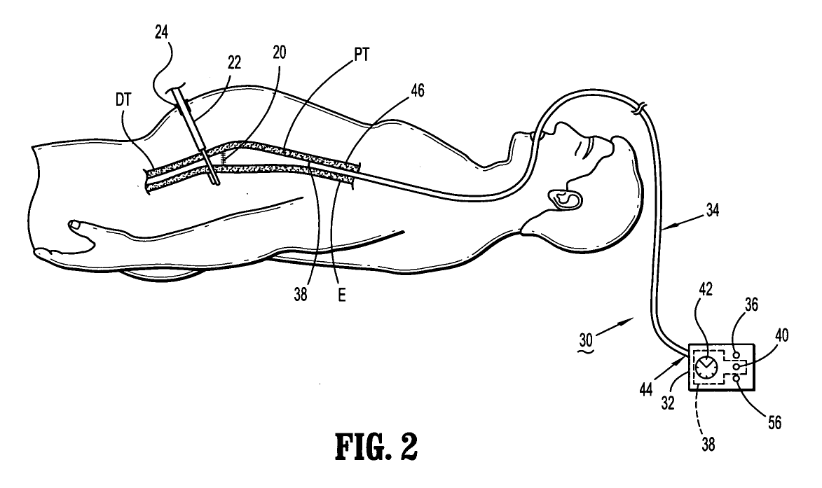

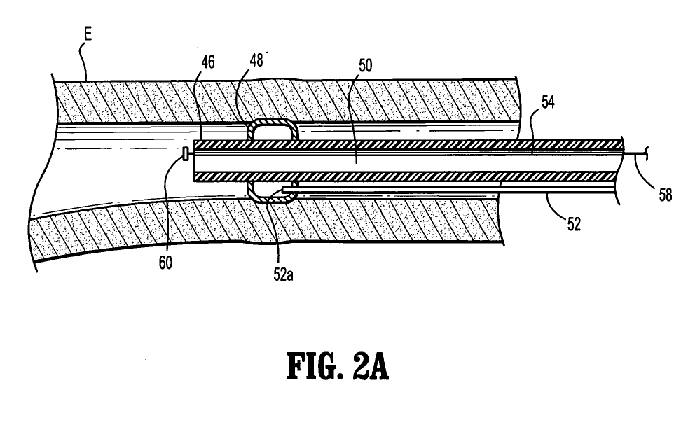

Referring now to FIG. 2, there is disclosed a first embodiment of a novel system, apparatus and method for accurately and instantaneously verifying the integrity of an anastomosis site. Integrity monitoring system 30 generally includes fluid source 32 and fluid conduit 34. Fluid source 32 is adapted to supply fluids under pressure (e.g., negative or positive pressure) to a tissue site. Fluid source 32 may be a conventional insufflation apparatus adapted to deliver CO2 gas at a predetermined pressure to the tissue site. Preferably, fluid source 32 includes pressure adjustment knob 36 to enable the user to selectively control and/or vary the fluid pressure being supplied to fluid conduit 34. Fluid source 32 further includes circuitry or computer logic to control, monitor and/or modify the various operating parameters of system 30. For example, fluid source 32 may incorporate pressure sensor means shown schematically as dashed lines 38 in FIG. 2 to permit the operator to monitor the fluid pressure adjacent the anastomosis site 20. Pressure sensor means 38 may incorporate circuitry or logic to calculate various parameters, including, e.g., the rate of pressure change, e.g., loss or decay or increase, at the anastomosis site, or simply detect and display real time pressure readings. Pressure sensor means 38 preferably incorporates a pressure transducer which is mounted to or extends within fluid conduit 34 as will be discussed hereinbelow. Pressure sensor means 38 may further include sensor adjustment knob 40 which provides a degree of control over the sensitivity of the pressure sensor means 38, i.e., to control the ability of the pressure sensor means 38 to detect gross or fine leaks. Pressure sensor means 38 is also provided with video display 42 which provides a visual indication in the form of a digital readout or gauge of an operating parameter of the system 30. Such parameters are inclusive of a real time pressure reading adjacent the anastomosis site 20, or, more preferably, a digital reading of the rate of loss or decay of the pressure adjacent the site. Pressure sensor means 38 may be a component of fluid source 32 or provided as a separate apparatus.

Fluid conduit 34 is in fluid communication with the pressure source 32 and incorporates proximal end 44 which is connected to the fluid source 32 and insertion end or distal end 46. As best depicted in FIG. 2A, fluid conduit 34 further includes occluding member 48 coaxially mounted about distal end 46. In the preferred embodiment, occluding member 48 is in the form of a balloon member adapted for expansion from a contracted state to an expanded state to substantially close off the esophageal tract. In the alternative, occluding member 48 may be a gel filled membrane or incorporate a septum or frusto-conical seal mounted about the outer surface of the fluid conduit 34. As a further alternative, distal end 46 of fluid conduit 34 may be sized to approximate the internal dimension of the tubular organ to substantially seal the lumen upon introduction therein. Fluid conduit 34 is preferably dimensioned for passage through the esophageal tract. Fluid conduit 34 may be provided with a filter to reduce the amount of impurities flowing through the fluid conduit 34.

With continued reference to FIG. 2A, fluid conduit 34 includes central lumen 50 and first and second supplemental lumens 52, 54. Central lumen 50 permits passage of pressurized fluid to the anastomosis site. First supplemental lumen 52 delivers the fluids used to expand occluding member or balloon member 48. First supplemental lumen 52 may be in fluid communication with fluid source 32 or in communication with an alternative fluid source. First supplemental lumen 52 may be a separate tube as shown or may be formed in the wall of fluid conduit 34. In the former arrangement, pressure source 32 may incorporate adjustment knob 56 (FIG. 2) to control fluid flow through first supplemental lumen 52 and exit port 52a to selectively expand occluding member 48. Second supplemental lumen 54 accommodates pressure detector lead 58 extending to pressure detector or transducer 60 mounted adjacent the distal end of fluid conduit 34. In the alternative, lumen 54 may be a pressure lumen with the pressure transducer 60 being within the external control unit. Second supplemental lumen 54 may include a separate tube or be defined in the wall of fluid conduit 54. Detector lead 58 is in electrical communication with the controller of pressure source 32.

In use, a gastric bypass surgery is performed resulting in an anastomosis site 20 located between a distal tubular section DT and proximal tubular section PT. Thereafter, a clamp, such as, for example, clamp 22 is inserted through a laparoscopic port 24 and used to clamp distal tubular section DT at a first tissue site disposed distal of anastomosis site 20. Thereafter, distal end 48 of fluid conduit 34 is introduced into the patient through esophagus E and into an area within proximal tubular section PT. Occluding member 48 is pressurized to expand the member 48 to substantially occlude the internal lumen of the proximal tubular section PT at a second tissue site on the opposed side of anastomosis site 20. Preferably, occluding member 48 provides a substantial seal to prevent escape of gases from the anastomosis site 20 through the occluding member 48 and through the lumen of the tubular organ.

Fluid source 32 is thereafter activated and adjusted by, e.g., means of pressure adjustment knob 42, to provide a predetermined level of pressure in the area adjacent anastomosis site 20 between the proximal and distal occluded tissue sites provided by occluding member 48 and clamp 22. Typically, a pressure level of approximately up to 200 mmHg is supplied during the evaluation of an anastomosis site 20 in a gastric bypass procedure. This pressure level is initially supplied to anastomosis site 20. No further amount of pressure needs to be applied to maintain the initial amount. Thereafter, sensor 42 is adjusted utilizing sensor adjustment knob 44 to achieve the desired sensitivity of the sensor 42. As noted above, pressure sensor means 38 evaluates the rate of pressure decay of the pressure provided to anastomosis site 20. This can be visually evaluated using visual display 42. A high or rapid rate of pressure decay indicates significant leakage at anastomosis site 20, which as noted above may require further surgical intervention. Conversely, a slow, or low, rate of pressure decay indicated by pressure sensor means 38 indicates an acceptable anastomosis 20. Thus, integrity monitoring system 30 provides a novel and accurate instrument for monitoring an anastomosis site 20 constructed during a gastric bypass surgical procedure.

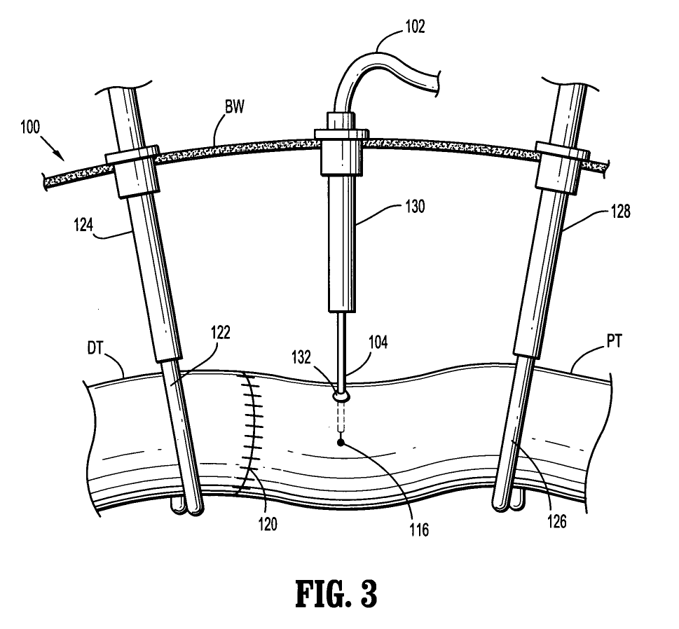

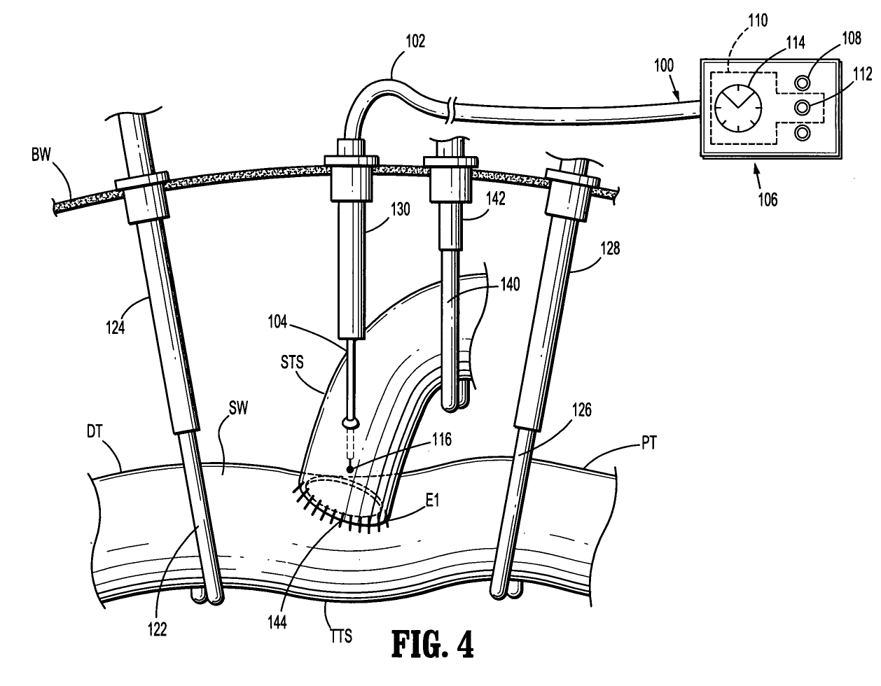

Referring now to FIGS. 3 and 4, there is disclosed an alternative integrity monitoring system 100 for use in monitoring an anastomosis site. Integrity monitoring system 100 includes fluid conduit 102 and hollow insertion needle 104 mounted to an end portion of the fluid conduit 102. Fluid conduit 102 is connected to pressure source 106 associated with integrity monitoring system 100. Insertion needle 104 is dimensioned to penetrate, pierce and/or puncture tissue. Preferably, insertion needle 104 includes a non-coring tip configured to penetrate tissue such that the resulting puncture wound can be readily healed. As with the previous embodiment, pressure source 106 is adapted to supply a predetermined amount of CO₂ through fluid conduit 102 and hollow insertion needle 104, and may be controlled via pressure adjustment knob 108.

Integrity monitoring system 100 further includes pressure sensor means indicated schematically by dashed lines 110. Pressure sensor means 110 is substantially similar to the pressure sensing means 38 described in connection with the embodiment of FIG. 2 and performs in similar manner to monitor the rate of pressure loss or, alternatively, real time pressure at the anastomosis site 20. Pressure sensor means 110 includes adjustment knob 112 and visual display 114. Pressure detector lead (not shown) and detector or transducer 116 are mounted to fluid conduit 102 and insertion needle 104 in the manner described hereinabove or by any other suitable conventional means. Preferably, pressure transducer or detector 116 extends to at least the distal end of insertion needle 104 as schematically shown in FIG. 3.

In use, an end to end anastomosis procedure is performed resulting in an anastomosis site 120 between a distal tubular section DT and a proximal tubular section PT. As with the previously disclosed end to end anastomosis procedure, a distal clamp 122 is inserted through a first port 124 in a body wall BW to substantially seal distal tubular section DT at a first site distal of anastomosis site 120. Similarly, a proximal clamp 126 is provided through a second port 128 in body wall BW to seal proximal tubular tissue section PT at a second tissue site proximal of anastomosis site 120. Third port 130 is provided through body wall BW and fluid conduit 102 and insertion needle 104 are introduced therethrough. Insertion needle 104 mounted to fluid conduit 102 is advanced through the tissue adjacent anastomosis site 120 such that non-coring tip of insertion needle 104 penetrates the tubular organ tissue. A sealant 132 may be provided about insertion needle 104 to seal the puncture site against any inadvertent leakage of CO₂. Once insertion needle 104 accesses the internal area of the anastomosis site, pressure source 106 is activated and the fluid pressure is adjusted using pressure adjustment knob 108. The pressure within the clamped tubular organ is detected with pressure detector 116, and the internal pressure is monitored by pressure sensor means 110 and viewed with visual display 114.

Referring now to FIG. 4, the use of integrity monitoring system 100 to verify the integrity of an end to side anastomosis will now be described. An end to side surgical procedure is performed such that a puncture is located in a side wall SW between distal tissue section DT and proximal tissue section PT in a first tubular tissue section TTS. An end E1 of a second tubular tissue section STS is connected through an anastomosis procedure to the side wall W. This particular procedure functions similar to that described above, however it is desirable to provide a third clamp 140 through a third port 142. Clamp 140 is configured to seal second tissue section STS proximally of anastomosis 144. Insertion needle 104 is inserted into second tissue section STS such that the non-coring tip penetrates second tissue section STS. Alternatively, insertion needle 104 may be inserted in to tubular tissue section TTS. Once insertion needle 104 has been properly positioned, the pressure source 106 can be activated and adjusted via adjustment knob 108 to provide an initial predetermined level of pressure to the anastomosis site 144. The pressure is monitored by sensor 116 after initial adjustments have been made with adjustment knob 112. The rate of pressure decay can be calculated via the circuitry or logic of pressure sensor means 110 and visually displayed on display 114.

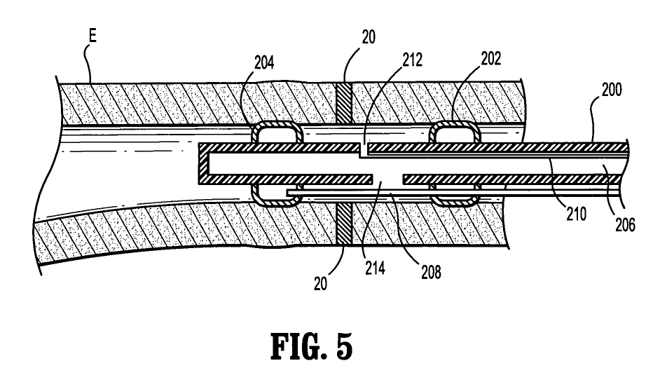

FIG. 5 illustrates an alternate embodiment of the present disclosure. In accordance with this embodiment, fluid conduit 200 includes first and second spaced expandable members 202, 204 at the insertion end of the fluid conduit 200. Fluid conduit 200 defines central lumen 206 for supplying fluid under pressure to the anastomosis site. Fluid conduit 200 further includes first and second supplemental lumens 208, 210. First lumen 208 delivers fluids to first and second expandable members 202, 204 to expand the members 202, 204. Second supplemental lumen 210 serves as the pressure lumen for detecting pressure loss. Second supplemental lumen 210 terminates in opening 212 defined in an intermediate wall portion of fluid conduit 200 between first and second expandable members 202,204. Second supplemental lumen 210 may be a pressure tube or incorporate a transducer as discussed hereinabove.

In use, fluid conduit 200 is introduced through the esophagus and advanced to position second expandable member 204 distal of the anastomosis site 20 and first expandable member 202 proximal of the anastomosis site. Expandable members 202, 204 are pressurized to substantially occlude the internal lumen at locations proximal and distal of the anastomosis site 20. Fluids are supplied under pressure through central lumen 206 to exit opening 214 disposed between first and second expandable members 202, 204. The pressure loss or rate of pressure loss adjacent the site and between expandable members 202, 204 is monitored with the pressure sensor means as discussed hereinabove. Expandable members 202, 204 may be balloon membranes filled with fluid or air.

It will be understood that various modifications may be made to the embodiments disclosed herein. For example, structure other than a sealing balloon may be provided at a distal end of the fluid conduit to facilitate sealing within the esophagus. Additionally, as noted above, the pressure source and sensors of the integrity checking device may be provided as a single unit or maybe provided separately. Further in the particular embodiment utilizing an insertion needle various other relatively a traumatic tips may be provided to facilitate puncturing the tubular tissue sections while allowing for relatively ease of healing and little loss of backflow air pressure. Still further, the sensor may be connected to other peripheral devices such as, for example, computers databases etc. to record and evaluate the pressure lost data. Therefore, the above description should not be construed as limiting, but merely as exemplifications of particular embodiments. Those skilled in the art will envision other modifications within the scope and spirit of the claims appended hereto.

What is claimed is:

1. A system for monitoring the integrity of an anastomosis of first and second tubular organ sections, which comprises:
a pressure source for supplying fluids under pressure;
first and second occluding members dimensioned for sealing a tubular organ section at first and second tissue sites adjacent respective opposed sides of an anastomosis site;
a fluid conduit connectable to the pressure source and being dimensioned for passage into the tubular organ section, the fluid conduit adapted to deliver the fluids to the tubular organ sections between the first and second tissue sites; and
a pressure sensor adapted to detect fluid pressure change adjacent the anastomosis site.

2. The system according to claim 1 wherein at least one of the first and second occluding members includes a clamp, the clamp being adapted for positioning about the tubular organ section.

3. The system according to claim 2 wherein each of the first and second occluding members includes a clamp, the clamps being adapted for positioning about the tubular organ section adjacent the first and second tissue sites.

4. The system according to claim 1 wherein the fluid conduit is adapted for passage through a lumen of the tubular organ section.

5. The system according to claim 4 wherein the fluid conduit includes an insertion end portion, the insert end portion adapted to form a substantial seal within the lumen of the tubular organ section adjacent one of the first and second tissue sites.

6. The system according to claim 5 wherein the fluid conduit includes an expandable member mounted adjacent the insertion end portion thereof, the expandable member being expandable to form a substantial seal within the lumen of the tubular organ section.

7. The system according to claim 6 wherein the expandable member includes a balloon member.

8. The system according to claim 6 wherein the pressure sensor is mounted to the fluid conduit.

9. The system according to claim 1 wherein the fluid conduit includes an insertion needle associated therewith, the insertion needle adapted to penetrate the tubular organ section to access the internal lumen thereof.

10. The system according to claim 9 wherein the pressure sensor is mounted to the fluid conduit.

11. The system according to claim 1 including a controller associated with the pressure sensor, the controller including logic to measure a rate of pressure change adjacent the anastomosis site.

12. The system according to claim 1 wherein the pressure sensor is adapted for positioning within the tubular organ section.

13. The system according to claim 1 including means for adjusting the sensitivity of the pressure sensor.

14. A method for monitoring the integrity of an anastomosis procedure, comprising the steps of:
sealing a tubular organ section at first and second tissue sites adjacent respective opposed sides of an anastomosis site;
introducing fluids adjacent the anastomosis site between the first and second tissue sites; and
monitoring pressure change adjacent the anastomosis site with a pressure sensor.

15. The method as recited in claim 14 wherein the step of monitoring includes introducing the pressure sensor within the tubular organ section between the first and second tissue sites.

16. The method as recited in claim 14 wherein the step of monitoring includes monitoring pressure loss.

17. The method as recited in claim 14 wherein the step of monitoring includes monitoring a rate of pressure loss.

18. The method as recited in claim 14 wherein the step of sealing includes applying a clamp adjacent at least one of the first and second tissue sites.

19. The method as recited in claim 14 wherein the step of sealing includes applying clamps adjacent each of the first and second tissue sites.

20. The method as recited in claim 14 wherein the step of introducing fluids includes positioning a fluid conduit into a lumen at the tubular organ section between the first and second tissue sites, the fluid conduit in fluid communication with a pressure source and adapted to deliver fluids under pressure to the tubular organ section.

21. The method as recited in claim 20 wherein the step of sealing includes positioning an end portion of the fluid conduit within the lumen of the tubular organ section and forming a substantial seal within the lumen of the tubular organ section with the end portion.

22. The method as recited in claim 21 wherein the end portion of the fluid conduit has an expandable member coaxially mounted thereabout and wherein the step of sealing includes expanding the expandable member to form a substantial seal within the lumen of the tubular organ section.

23. The method as recited in claim 22 wherein the expandable member includes a balloon member and wherein the step of sealing includes introducing fluids into the balloon member to expand the balloon member to form the substantial seal within the lumen.

24. The method as recited in claim 20 wherein the fluid conduit includes a pressure sensor and wherein the step of monitoring includes positioning the pressure sensor within the tubular organ section between the first and second tissue sites.
